(19) United States
(12) Patent Application Publication
(10) Pub. No.: US 2021/0340637 A1
(43) Pub. Date: Nov. 4, 2021

(54) METHOD OF PRODUCING JUICE FROM SUGAR-CONTAINING RAW MATERIALS

(71) Applicant: IDEPS GmbH, Vienna (AT)

(72) Inventors: Oleksandr KOVAL, Odessa (UA); Dmytro TRETYAKOV, City Brovary (UA); Tamila SHEIKO, City Vyshneve (UA); Serhii HATSENKO, Odessa (UA); Oswald BRANDSTETTER, Bratislava (SK)

(21) Appl. No.: 17/373,931

(22) Filed: Jul. 13, 2021

Related U.S. Application Data
(63) Continuation-in-part of application No. 16/462,689, filed on May 21, 2019, now Pat. No. 11,089,784.

(30) Foreign Application Priority Data
Nov. 28, 2016 (UA) A 2016 12062

(51) Int. Cl. C13B 10/14; A23L 27/10; C13B 10/02

(52) U.S. Cl. CPC C13B 10/14; A23V 2002/00; C13B 10/025; A23L 27/11

(57) ABSTRACT

A method for obtaining juice from sugar-containing raw materials is disclosed. The method includes grinding and extracting sugar by adding a reagent causing a partial destruction of the non-sugar polymer chains within the raw material. The inorganic reagent includes sodium peroxidesulfate (i.e., sodium persulfate), ammonium peroxydisulfate (i.e., ammonium persulfate), or potassium peroxydisulfate (i.e., potassium persulfate), not less than 0.50% weight, and sodium hydroxide or potassium hydroxide, not less than 0.50% weight. The reagent is added to the sugar-containing raw material, the collection of juice, and/or any pulp collection water (e.g., in the beet industry). The total reagent amount added ranges between 0.001-0.02% weight of the raw materials. The mixture is then incubated for 15-20 minutes at a temperature ranging from 25 to 75° C.

### CROSS-REFERENCE TO RELATED APPLICATIONS

This patent application is a Continuation-in-Part of U.S. patent application Ser. No. 16/462,689 filed on May 19, 2019, which is a National stage application from PCT application PCT/UA2016/000147 filed on Dec. 19, 2016, which claims priority to Ukrainian application a 2016 12062 filed on Dec. 28, 2016, all of which are incorporated fully herein by reference.

### FIELD OF THE INVENTION

The invention relates to the food industry, namely to the technology of sugar production via extraction of sucrose. The invention may be used in sugar factories for processing of, e.g., sugar cane and beet.

### BACKGROUND OF THE INVENTION

Known methods of obtaining juice from sugar-containing raw materials include the grinding of raw materials, sucrose extraction from the ground material, purification, thickening, and boiling.

A known method for the treatment of sugar-containing raw materials exists, which discloses the use of various acids, as well as sodium and phosphorus salts up to a pH of 5 (See U.S. Pat. No. 6,068,869, Method of producing a stabilized sugar cane juice product).

Another method of extracting sucrose from beet chips is known, which includes pre-treatment and countercurrent extraction of sulfite and pulp water. The pre-treatment of beet chips according to the known method is carried out with peroxyacetic acid (See FR 2807065 A1, Process for the disinfection of sugar solutions comprising the treatment of cossettes with a peracetic acid solution).

The disadvantages of these known methods are low efficiency of their action. In addition, these substances are partially accumulated in semi-products of sugar-beet production, keeping sugars from crystalizing and thereby reducing the quality of sugar products.

### SUMMARY OF THE INVENTION

The invention is based on the task of partial destruction of polysaccharide polymer chains with glycoside bonds—dextran, pectin, high molecular weight compounds (hereinafter non-sugars), transferred into the juice from sugar-containing raw materials, and which are the cause of reducing the quality of juice, as well as the cause of foaming at all stages of sugar beet production, an inorganic reagent that hydrolyzes and destructurizes the above substances into shorter (simple) chains with smaller molecular weight, and also produces a disinfecting effect. As a result, there is an increase in the quality of semi-products and sugar, as well as increasing its output.

The problem is solved by the fact that the method for obtaining juice from sugar-containing raw materials, including grinding of raw materials, sucrose extraction from it, further purification of juice, its thickening and boiling sugar. For partial destruction of the non-sugar polymer chains on the raw material and to the water that is used at the stage of juice extraction, a sulfur-containing inorganic reagent is added. The inorganic reagent specifically contains sodium peroxidesulfate (i.e., sodium persulfate), ammonium peroxydisulfate (i.e., ammonium persulfate), and/or potassium peroxydisulfate (i.e., potassium persulfate), not less than 0.50%, and sodium hydroxide or potassium hydroxide, not less than 0.50%, is added. The reagent (e.g., within a solution) is added to the sugar-containing raw material, the collection of juice, and/or any pulp collection water (e.g., in the beet industry). The total reagent amount added ranges between 0.001-0.02% weight of the raw materials. The mixture is then incubated for 15-20 minutes at a temperature ranging from 25 to 75° C.

The general properties of sodium peroxodisulfate, potassium peroxodisulfate, and ammonium peroxodisulfate, which provide the effectiveness of the present invention, are as follows:
are salts of peroxodic acid (H2S2O8);
are strong oxidizing agents;
have a disinfectant effect;
are sources of free oxygen (O2), peroxide (H2O2) and hydroperoxide groups (HO2-);
are sources of the sulfate radical (SO4-) and contribute to the formation of the hydroxyl radical (HO—).

Due to such properties, compositions based on sodium peroxydisulfate (i.e., sodium persulfate), potassium peroxydisulfate (i.e., potassium persulfate), and ammonium peroxydisulfate (i.e., ammonium persulfate) allow to obtain the effect of partial destruction of non-sugar polymer chains within sugar-containing raw materials or non-sugars within water, and they are used in the juice extraction stage. In addition, these compositions contribute a secondary bactericidal effect on the extracted product.

### DETAILED DESCRIPTION OF THE PREFERRED EMBODIMENT

Sequence of Method Steps.

A method is disclosed for obtaining juice from sugar-containing raw materials, including the grinding of raw materials, the extraction of sucrose from it. For partial destruction of non-sugar polymer chains on the surface of raw materials, inorganic reagent based on sodium peroxodisulfate, and/or ammonium peroxydisulfate, and/or potassium peroxydisulfate not less than 0.50% and sodium hydroxide or potassium hydroxide not less than 0.50% is added to juice and press water (in sugar beet industry) which is applied to raw materials, is introduced into the juice collector and press water collector (in sugar beet industry) in a total amount of 0.001 to 0.02% by weight of raw material, at a temperature ranging from 25 to 75° C. and allowed to stand for 15-20 minutes to undergo a partial degradation of polysaccharide polymer chains. Then further purification of the juice, its thickening and boiling sugar is carried out.

### EXAMPLES OF THE METHOD OF OBTAINING JUICE FROM SUGAR-CONTAINING RAW MATERIALS

Example 1. Sugar-Containing Raw Material was Crushed, Sucrose was Extracted from it and juice was obtained. The juice was purified, thickened and the sugar was boiled away.

Example 2. Processed sugar-containing raw material (sugar cane) with an inorganic preparation, crushed it, extracted the juice. For partial destruction of polymeric chains of non-sugars an inorganic reagent based on sodium persulfate, not less than 0.50% and sodium hydroxide, not less than 0.50% was added to the juice and applied to raw material after washing, It was introduced into the juice collector in a total amount of 0.0001-0.001% by weight of raw material, at a temperature ranging from 25 to 45° C. and was kept for 15-20 minutes for the process of partial destruction of polysaccharide polymer chains of non-saccharides. Further juice purification, thickening and sugar boiling were carried out.

Example 3. Example 3 is performed similarly to Example 2. An inorganic reagent based on sodium persulfate, not less than 0.50% and sodium hydroxide, not less than 0.50% was added in total amount of 0.001-0.02% to mass of raw material (sugarcane) at temperature in range from 25 to 45° C. and was kept for 15-20 minutes for passing of partial destruction process of polysaccharide non-sugar polymer chains. Further juice purification, thickening and sugar boiling were carried out.

Example 4. Example 4 is performed similarly to Example 2. A inorganic reagent based on sodium persulfate, not less than 0.50% and sodium hydroxide, not less than 0.50% was added in total amount of 0.025-0.05% to mass of raw material (sugarcane), at temperature in range from 25 to 45° C. and was kept for 15-20 minutes for passing of process of partial destruction of polysaccharide polymer chains of non-sugars. Further juice purification, thickening and sugar boiling were carried out.

Example 5. Processed sugar-containing raw material (sugar cane) with an inorganic preparation, crushed it, extracted the juice. For partial destruction of non-sugar polymer chains an inorganic reagent based on ammonium persulfate,

not less than 0.50% and sodium hydroxide, not less than 0.50% was added to the juice and applied to raw material after washing, It was introduced into the juice collector in a total amount of 0.0001-0.001% by weight of raw material, at a temperature ranging from 25 to 45° C. and was kept for 15-20 minutes for the process of partial destruction of polysaccharide polymer chains of non-saccharides. Further juice purification, thickening and sugar boiling were carried out.

Example 6. Example 6 is performed similarly to Example 5. An inorganic reagent based on ammonium persulfate, not less than 0.50% and sodium hydroxide, not less than 0.50% was added in total amount of 0.001-0.02% to mass of raw material (sugar cane) at temperature in range from 25 to 45° C. and was kept for 15-20 minutes for passing the process of partial destruction of polysaccharide polymer chains of non-sugars. Further juice purification, thickening and sugar boiling were carried out.

Example 7. Example 7 is performed similarly to Example 5. An inorganic reagent based on ammonium persulfate, not less than 0.50% and sodium hydroxide, not less than 0.50% was added in total amount of 0.025-0.05% to mass of raw material (sugar cane) at temperature in range from 25 to 45° C. and was kept for 15-20 minutes for passage of partial destruction process of polysaccharide polymer chains of non-saccharides. Further juice purification, thickening and sugar boiling were carried out.

Example 8. Sugar-containing raw material (sugar cane) was treated with inorganic preparation, crushed, juice was extracted. For partial destruction of non-sugar polymer chains an inorganic reagent based on potassium persulfate not less than 0.50% and potassium hydroxide not less than 0.50% was added to the juice and applied to the raw material after washing, It was introduced into the juice collector in a total amount of 0.0001-0.001% by weight of raw material at a temperature ranging from 25 to 45° C. and was kept for 15-20 minutes for the process of partial destruction of polysaccharide polymer chains of non-saccharides. Further juice purification, thickening and sugar boiling were carried out.

Example 9. Example 9 is performed similarly to Example 8. An inorganic reagent based on potassium persulfate at least 0.50% and potassium hydroxide at least 0.50% was added in total amount of 0.001-0.02% to mass of raw material (sugar cane), at temperature in range from 25 to 45° C. and was kept for 15-20 minutes for passing process of partial destruction of polysaccharide non-sugar polymer chains. Further juice purification, thickening and sugar boiling were carried out.

Example 10. Example 10 is performed similarly to Example 8. A inorganic reagent based on potassium persulfate at least 0.50% and potassium hydroxide at least 0.50% was added in total amount of 0.025-0.05% to mass of raw material (sugar cane) at temperature in range from 25 to 45° C. and was kept for 15-20 minutes for passage of partial destruction process of polysaccharide non-sugars polymer chains. Further juice purification, thickening and sugar boiling were carried out.

Example 11. Processed sugar-containing raw material (sugar beet) with inorganic preparation, milled it, extracted juice. For partial destruction of non-sugar polymer chains an inorganic reagent based on sodium persulfate, not less than 0.50% and sodium hydroxide, not less than 0.50% was added to the juice and applied to raw material after washing, It was put in the juice collector and press water collector in total amount 0.0001-0.001% to mass of raw material at temperature in range from 45 to 75° C. and was kept 15-20 minutes for passing the process of partial destruction of polysaccharide polymer chains of non-saccharides. Further juice purification, thickening and sugar boiling were carried out.

Example 12. Example 12 is performed similarly to Example 11. An inorganic reagent based on sodium persulfate, not less than 0.50% and sodium hydroxide, not less than 0.50% was added in total amount of 0.001-0.02% to mass of raw material (sugar beet), at temperature in range from 45 to 75° C. and was kept for 15-20 minutes for passing of process of partial destruction of polysaccharide non-sugar polymer chains. Further juice purification, thickening and sugar boiling were carried out.

Example 13. Example 13 is performed similarly to Example 11. A inorganic reagent based on sodium persulfate, not less than 0.50% and sodium hydroxide, not less than 0.50% was added in a total amount of 0.025-0.05% to the mass of raw material (sugar beet), at a temperature ranging from 45 to 75° C. and kept for 15-20 minutes to undergo a process of partial destruction of polysaccharide polymer chains of non-sugars. Further juice purification, thickening and sugar boiling were carried out.

Example 14. Processed sugar-containing raw material (sugar beet) with an inorganic preparation, milled it, extracted the juice. For partial destruction of non-sugar polymer chains an inorganic reagent based on ammonium persulfate, not less than 0.50% and sodium hydroxide, not less than 0.50% was added to the juice and applied to raw material after washing, It was put in the juice collector and press water collector in total amount 0.0001-0.001% to mass of raw material at temperature in range from 45 to 75° C. and was kept 15-20 minutes for passing the process of partial destruction of polysaccharide polymer chains of non-saccharides. Further juice purification, thickening and sugar boiling were carried out.

Example 15. Example 15 is performed similarly to Example 14. An inorganic reagent based on ammonium persulfate, not less than 0.50% and sodium hydroxide, not less than 0.50% was added in a total amount of 0.001-0.02% to mass of raw material (sugar beet), at a temperature ranging from 45 to 75° C. and was kept for 15-20 minutes to pass the process of partial destruction of polysaccharide non-sugar polymer chains. Further juice purification, thickening and sugar boiling were carried out.

Example 16. Example 16 is performed similarly to Example 14. An inorganic reagent based on ammonium persulfate, not less than 0.50% and sodium hydroxide, not less than 0.50% was added in a total amount of 0.025-0.05% to mass of raw material (sugar beet), at a temperature in the range from 45 to 75° C. and kept for 15-20 minutes for the process of partial destruction of polysaccharide polymer chains of non-sugars. Further juice purification, thickening and sugar boiling were carried out.

Example 17. Processed sugar-containing raw material (sugar beet) with an inorganic preparation, milled it, extracted the juice. For partial destruction of non-sugar polymer chains an inorganic reagent based on potassium persulfate not less than 0.50% and potassium hydroxide not less than 0.50% was added to the juice and applied to raw material after washing, It was introduced into the juice collector and press water collector in a total amount of 0.0001-0.001% by weight of raw materials at temperatures ranging from 45 to 75° C. and was kept for 15-20 minutes for the process of partial destruction of polysaccharide polymer chains of sugars. Further juice purification, thickening and sugar boiling were carried out.

Example 18. Example 18 is performed similarly to Example 17. An inorganic reagent based on potassium persulfate, not less than 0.50% and potassium hydroxide, not less than 0.50% was added in a total amount of 0.001-0.02% to mass of raw material (sugar beet), at a temperature ranging from 45 to 75° C. and kept for 15-20 minutes to undergo a partial destruction of polysaccharide non-sugar polymer chains. Further juice purification, thickening and sugar boiling were carried out.

Example 19. Example 19 is performed similarly to Example 17. A inorganic reagent based on potassium persulfate not less than 0.50% and potassium hydroxide not less than 0.50% was added in a total amount of 0.025-0.05% to mass of raw material (sugar beet), at temperature in range from 45 to 75° C. and kept for 15-20 minutes for passing of process of partial destruction of polysaccharide polymer chains of non-sugars. Further juice purification, thickening and sugar boiling were carried out.

Example 20. Inorganic reagent based on sodium persulfate at least 0.50% and sodium hydroxide at least 0.50% was added in total amount of 0.001-0.02% to mass of raw material (sugar cane) at temperature in range from 25 to 45° C. and was kept for 5-10 minutes for passing process of partial destruction of polysaccharide non-sugar polymer chains. Further juice purification, thickening and sugar boiling were carried out.

Example 21. Example 21 is performed similarly to Example 20. An inorganic reagent based on sodium persulfate, not less than 0.50% and sodium

hydroxide, not less than 0.50% was added in total amount of 0.001-0.02% to mass of raw material (sugar cane), at temperature in range from 25 to 45° C. and was kept for 15-20 minutes for passing of partial degradation process of polysaccharide polymer chains of non-sugars. Further juice purification, thickening and sugar boiling were carried out.

Example 22. Example 22 is performed similarly to Example 20. An inorganic reagent based on sodium persulfate, not less than 0.50% and sodium hydroxide, not less than 0.50% was added in total amount of 0.001-0.02% to mass of raw material (sugar cane), at temperature in range from 25 to 45° C. and was kept for 25-30 minutes for passing of partial degradation process of polysaccharide polymer chains of non-sugars. Further juice purification, thickening and sugar boiling were carried out.

Example 23. Inorganic reagent based on ammonium persulfate, not less than 0.50% and sodium hydroxide, not less than 0.50% was added in total amount of 0.001-0.02% to mass of raw material (sugar cane) at temperature in range from 25 to 45° C. and was kept for 5-10 minutes for passing the process of partial destruction of polysaccharide polymer chains of non-sugars. Further juice purification, thickening and sugar boiling were carried out.

Example 24. Example 24 is performed similarly to Example 23. An inorganic reagent based on ammonium persulfate, not less than 0.50% and sodium hydroxide, not less than 0.50% was added in total amount of 0.001-0.02% to mass of raw material (sugar cane), at temperature in range from 25 to 45° C. and was kept for 15-20 minutes for passing of partial degradation process of polysaccharide non-sugar polymer chains. Further juice purification, thickening and sugar boiling were carried out.

Example 25. Example 25 is performed similarly to Example 23. An inorganic reagent based on ammonium persulfate, not less than 0.50% and sodium hydroxide, not less than 0.50%, was added in a total amount of 0.001-0.02% to mass of raw material (sugar cane), at a temperature in the range from 25 to 45° C. and kept for 25-30 minutes to undergo partial degradation of polysaccharide non-saccharide polymer chains. Further juice purification, thickening and sugar boiling were carried out.

Example 26. Inorganic reagent based on potassium persulfate not less than 0.50% and potassium hydroxide not less than 0.50% was added in total amount of 0.001-0.02% to mass of raw material (sugar cane) at temperature in range from 25 to 45° C. and was kept for 5-10 minutes for passing the process of partial destruction of polysaccharide polymer chains of non-sugars. Further juice purification, thickening and sugar boiling were carried out.

Example 27. Example 27 is performed similarly to Example 26. An inorganic reagent based on potassium persulfate at least 0.50% and potassium hydroxide at least 0.50% was added in total amount of 0.001-0.02% to mass of raw material (sugar cane), at temperature in range from 25 to 45° C. and was kept

for 15-20 minutes for passing process of partial destruction of polysaccharide non-sugar polymer chains. Further juice purification, thickening and sugar boiling were carried out.

Example 28. Example 28 is performed similarly to Example 26. An inorganic reagent based on sodium persulfate, not less than 0.50% and sodium hydroxide, not less than 0.50% was added in total amount of 0.001-0.02% to mass of raw material (sugar cane), at temperature in range from 25 to 45° C. and was kept for 25-30 minutes for passing of process of partial destruction of polysaccharide polymer chains of non-sugars. Further juice purification, thickening and sugar boiling were carried out.

Example 29. Inorganic reagent based on sodium persulfate, not less than 0.50% and sodium hydroxide, not less than 0.50% were added in total amount of 0.001-0.02% to mass of raw material (sugar beet) at temperature in range from 45 to 75° C. and kept for 5-10 minutes for passing the process of partial destruction of polysaccharide polymer chains of non-sugars. Further juice purification, thickening and sugar boiling were carried out.

Example 30. Example 30 is performed similarly to Example 29. A inorganic reagent based on sodium persulfate, not less than 0.50% and sodium hydroxide, not less than 0.50%, was added in a total amount of 0.001-0.02% to the mass of raw material (sugar beet), at a temperature in the range from 45 to 75° C. and kept for 15-20 minutes to undergo a partial degradation of polysaccharide polymer chains of non-sugars. Further juice purification, thickening and sugar boiling were carried out.

Example 31. Example 31 is performed similarly to Example 29. A inorganic reagent based on sodium persulfate, not less than 0.50% and sodium hydroxide, not less than 0.50%, was added in a total amount of 0.001-0.02% to the mass of raw material (sugar beet), at temperature in the range from 45 to 75° C. and kept 25-30 minutes for the process of partial destruction of polysaccharide non-sugar polymer chains. Further juice purification, thickening and sugar boiling were carried out.

Example 32. Inorganic reagent based on ammonium persulfate, not less than 0.50% and sodium hydroxide, not less than 0.50%, was added in a total amount of 0.001-0.02% to the mass of raw materials (sugar beet) at a temperature ranging from 45 to 75° C. and kept 5-10 minutes for the process of partial destruction of polysaccharide chains of non-sugars. Further juice purification, thickening and sugar boiling were carried out.

Example 33. Example 33 is performed similarly to Example 32. An inorganic reagent based on ammonium persulfate, not less than 0.50% and sodium hydroxide, not less than 0.50% was added in a total amount of 0.001-0.02% to mass of raw material (sugar beet), at temperature in range from 45 to 75° C. and kept 15-20 minutes for passing process of partial destruction of polysaccharide non-saccharide polymer chains. Further juice purification, thickening and sugar boiling were carried out.

Example 34. Example 34 is performed similarly to Example 32. An inorganic reagent based on ammonium persulfate, not less than 0.50% and sodium hydroxide, not less than 0.50% was added in a total amount of 0.001-0.02% to mass of raw material (sugar beet), at temperature in range from 45 to 75° C. and was kept 25-30 minutes for passing of partial degradation process of polysaccharide polymer chains of non-sugars. Further juice purification, thickening and sugar boiling were carried out.

Example 35. Inorganic reagent based on potassium persulfate, not less than 0.50% and potassium hydroxide, not less than 0.50% was added in a total amount of 0.001-0.02% to mass of raw materials (sugar beet) at a temperature ranging from 45 to 75° C. and kept for 5-10 minutes for the process of partial destruction of polysaccharide non-sugar polymer chains. Further juice purification, thickening and sugar boiling were carried out.

Example 36. Example 36 is performed similarly to Example 35. An inorganic reagent based on potassium persulfate, not less than 0.50% and potassium hydroxide, not less than 0.50% was added in a total amount of 0.001-0.02% to mass of raw material (sugar beet), at temperature in range from 45 to 75° C. and kept for 15-20 minutes for passing of partial degradation process of polysaccharide polymer chains of non-sugars. Further juice purification, thickening and sugar boiling were carried out.

Example 37. Example 37 is performed similarly to Example 35. A inorganic reagent based on potassium persulfate not less than 0.50% and potassium hydroxide not less than 0.50% was added in a total amount of 0.001-0.02% to mass of raw material (sugar beet), at a temperature ranging from 45 to 75° C. and kept 25-30 minutes for the process of partial destruction of polysaccharide non-sugar polymer chains. Further juice purification, thickening and sugar boiling were carried out.

Results of Experimentation.

The results of the studies on examples 1-19 are provided in Table 1. The dependence of physico-chemical parameters of juice on the amount of inorganic reagent added, under conditions of juice treatment for 15-20 minutes, are provided in the table. Table 2 relates to examples 20-34 and provides the dependence of physico-chemical parameters of juice on the duration of treatment with inorganic reagent in a total amount of 0.001-0.02% relative to the mass of raw material (cane and/or beet).

From the above examples, the best resulting partial degradation of non-sugar polymer chains is observed in Examples 3, 6, 9, 21, 24, and 27 for sugar cane, and in Examples 12, 15, 18, 30, 33, and 36 for beet sugar.

The technical result from the implementation of the method consists in improving the physical and chemical parameters of the juice, juice disinfection, reducing foaming of semi-products, which will eliminate the addition of a large number of additional chemicals and defoamers, which remain in the semi-products, keeping part of sucrose from crystallization, and accumulate in molasses, reducing its quality. This will ultimately improve the quality of sugar and increase the yield of sugar.

Thus, given the positive result when using this method of obtaining juice from sugar-containing products, it can be recommended for use in the food industry, in particular in the technology of sugar production in the extraction of sucrose.

The description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

TABLE 1
Dependence of juice physicochemical parameters on the total amount of inorganic
reagent consumed under juice treatment conditions of 15-20 minutes

| | | Physicochemical parameters of juice | | | Loss of | | |
|---|---|---|---|---|---|---|---|
| | | | | High- | sucrose | | |
| | | | | molecular | from | | |
| | Total | | | weight | decomposi- | | |
| | consumption of | | | compounds | tion, % by | Lactic | |
| | inorganic reagent, | | | content, % | weight of | acid | |
| | % by weight of | Dextran | Pectin | per 100 | raw | content | |
| Example | raw material | content, % | content, % | solids | material | mg/l | pH |

Sugar cane juice
Inorganic reagent based on sodium persulfate, not less than 0.50%,
and sodium hydroxide, not less than 0.50%

| | | | | | | | |
|---|---|---|---|---|---|---|---|
| 1 | Without adding | 0.18 | 0.79 | 16 | 0.02 | 892 | 4.8 |
| | an inorganic | | | | | | |
| | reagent | | | | | | |
| 2 | 0.0001-0.001% | 0.12 | 0.75 | 14 | 0.02 | 754 | 5.0 |
| 3 | 0.001-0.02% | 0.05 | 0.42 | 7 | 0.02 | 413 | 5.4 |
| 4 | 0.025-0.05% | 0.03 | 0.39 | 6 | 0.03 | 389 | 5.9 |

Inorganic reagent based on ammonium persulfate, not less than 0.50%
and sodium hydroxide, not less than 0.50%

| | | | | | | | |
|---|---|---|---|---|---|---|---|
| 1 | Without adding | 0.18 | 0.79 | 16 | 0.02 | 892 | 4.8 |
| | an inorganic | | | | | | |
| | reagent | | | | | | |
| 5 | 0.0001-0.001% | 0.11 | 0.76 | 16 | 0.02 | 654 | 4.9 |
| 6 | 0.001-0.02% | 0.06 | 0.47 | 8 | 0.02 | 458 | 5.2 |
| 7 | 0.025-0.05% | 0.06 | 0.42 | 8 | 0.02 | 438 | 5.3 |

Inorganic reagent based on potassium persulfate, not less than 0.50%
and sodium hydroxide, not less than 0.50%

| | | | | | | | |
|---|---|---|---|---|---|---|---|
| 1 | Without adding | 0.18 | 0.79 | 16 | 0.02 | 892 | 4.8 |
| | an inorganic | | | | | | |
| | reagent | | | | | | |
| 8 | 0.0001-0.001% | 0.14 | 0.43 | 12 | 0.02 | 712 | 4.9 |
| 9 | 0.001-0.02% | 0.10 | 0.39 | 10 | 0.02 | 482 | 5.2 |
| 10 | 0.025-0.05% | 0.08 | 0.37 | 9 | 0.022 | 412 | 5.3 |

Diffusion juice of sugar beet production
Inorganic reagent based on sodium persulfate, not less than 0.50%,
and sodium hydroxide, not less than 0.50%

| | | | | | | | |
|---|---|---|---|---|---|---|---|
| 1 | Without adding | 0.15 | 0.979 | 12 | 0.02 | 554 | 5.4 |
| | an inorganic | | | | | | |
| | reagent | | | | | | |
| 11 | 0.0001-0.001% | 0.09 | 0.67 | 11 | 0.02 | 385 | 5.8 |
| 12 | 0.001-0.02% | 0.06 | 0.593 | 9 | 0.02 | 322 | 6.0 |
| 13 | 0.025-0.05% | 0.06 | 0.512 | 7 | 0.03 | 296 | 6.2 |

Inorganic reagent based on ammonium persulfate, not less than 0.50%,
and sodium hydroxide, not less than 0.50%

| | | | | | | | |
|---|---|---|---|---|---|---|---|
| 1 | Without adding | 0.15 | 0.979 | 12 | 0.02 | 554 | 5.4 |
| | an inorganic | | | | | | |
| | reagent | | | | | | |
| 14 | 0.0001-0.001% | 0.11 | 0.734 | 10 | 0.02 | 356 | 5.6 |
| 15 | 0.001-0.02% | 0.08 | 0.612 | 9 | 0.02 | 324 | 5.8 |
| 16 | 0.025-0.05% | 0.07 | 0.532 | 9 | 0.02 | 308 | 6.0 |

Inorganic reagent based on potassium persulfate, not less than 0.50%,
and sodium hydroxide, not less than 0.50%

| | | | | | | | |
|---|---|---|---|---|---|---|---|
| 1 | Without adding | 0.15 | 0.979 | 12 | 0.02 | 554 | 5.4 |
| | an inorganic | | | | | | |
| | reagent | | | | | | |
| 17 | 0.0001-0.001% | 0.09 | 0.698 | 10 | 0.02 | 397 | 5.7 |
| 18 | 0.001-0.02% | 0.06 | 0.627 | 8 | 0.02 | 324 | 5.9 |
| 19 | 0.025-0.05% | 0.06 | 0.574 | 7 | 0.02 | 296 | 6.1 |

TABLE 2
Dependence of juice physicochemical parameters on the duration of treatment with a chemical reagent
in a total amount of 0.001-0.02% to the mass of raw material (sugar cane and sugar beet)

| | | Physical and chemical parameters of juice | | | | | |
|---|---|---|---|---|---|---|---|
| | | | | High- | | | |
| | | | | molecular | | | |
| | | | | weight | Loss of | | |
| | | | | compounds | sucrose from | | |
| | | | | content, % | decomposition, | Lactic acid | |
| | Processing | Dextran | Pectin | per 100 | % by weight of | content, | |
| Example | time, min | content, % | content, % | solids | raw material | mg/l | pH |

Sugar cane juice
Inorganic reagent based on sodium persulfate, not less than 0.50%,
and sodium hydroxide, not less than 0.50%

| | | | | | | | |
|---|---|---|---|---|---|---|---|
| 20 | 5-10 | 0.14 | 0.628 | 12 | 0.02 | 785 | 4.9 |
| 21 | 15-20 | 0.05 | 0.42 | 7 | 0.02 | 413 | 5.4 |
| 22 | 25-30 | 0.04 | 0.37 | 5 | 0.05 | 369 | 5.4 |

Inorganic reagent based on ammonium persulfate, not less than 0.50%,
and sodium hydroxide, not less than 0.50%

| | | | | | | | |
|---|---|---|---|---|---|---|---|
| 23 | 5-10 | 0.12 | 0.606 | 11 | 0.02 | 754 | 5.0 |
| 24 | 15-20 | 0.06 | 0.470 | 8 | 0.02 | 458 | 5.2 |
| 25 | 25-30 | 0.05 | 0.302 | 7 | 0.03 | 316 | 5.4 |

Inorganic reagent based on potassium persulfate, not less than 0.50%,
and sodium hydroxide, not less than 0.50%

| | | | | | | | |
|---|---|---|---|---|---|---|---|
| 26 | 5-10 | 0.14 | 0.615 | 12 | 0.02 | 712 | 5.1 |
| 27 | 15-20 | 0.10 | 0.390 | 10 | 0.02 | 482 | 5.2 |
| 28 | 25-30 | 0.06 | 0.346 | 8 | 0.03 | 356 | 5.5 |

Diffusion juice of sugar beet production
Inorganic reagent based on sodium persulfate, not less than 0.50%,
and sodium hydroxide, not less than 0.50%

| | | | | | | | |
|---|---|---|---|---|---|---|---|
| 29 | 5-10 | 0.14 | 0.780 | 11 | 0.02 | 415 | 5.6 |
| 30 | 15-20 | 0.06 | 0.593 | 9 | 0.02 | 322 | 6.0 |
| 31 | 25-30 | 0.05 | 0.494 | 9 | 0.04 | 236 | 6.0 |

Inorganic reagent based on ammonium persulfate, not less than 0.50%,
and sodium hydroxide, not less than 0.50%

| | | | | | | | |
|---|---|---|---|---|---|---|---|
| 32 | 5-10 | 0.12 | 0.755 | 12 | 0.02 | 478 | 5.8 |
| 33 | 15-20 | 0.08 | 0.612 | 9 | 0.02 | 324 | 5.8 |
| 34 | 25-30 | 0.07 | 0.536 | 7 | 0.03 | 299 | 6.0 |

Inorganic reagent based on potassium persulfate, not less than 0.50%,
and sodium hydroxide, not less than 0.50%

| | | | | | | | |
|---|---|---|---|---|---|---|---|
| 35 | 5-10 | 0.14 | 0.769 | 11 | 0.02 | 437 | 5.9 |
| 36 | 15-20 | 0.06 | 0.627 | 8 | 0.02 | 324 | 5.9 |
| 37 | 25-30 | 0.06 | 0.567 | 7 | 0.03 | 278 | 6.2 |

What is claimed is:

1. A method for obtaining sucrose juice from a sugar-containing product, comprising:
grinding of the sugar-containing product, and
extracting sucrose juice from the sugar-containing product by adding a solution, the solution comprising an inorganic sulfur-containing reagent.

2. The method of claim 1, wherein the inorganic sulfur-containing reagent comprises sodium persulfate, not less than 0.5% wt, and sodium hydroxide, not less than 0.5% wt.

3. The method of claim 1, wherein the inorganic sulfur-containing reagent comprises ammonium persulfate, not less than 0.5% wt, and sodium hydroxide, not less than 0.5% wt.

4. The method of claim 1, wherein the inorganic sulfur-containing reagent comprises potassium persulfate, not less than 0.5% wt, and potassium hydroxide, not less than 0.5% wt.

5. The method of claim 1, wherein the sugar-containing product is sugar cane.

6. The method of claim 1, wherein the sugar-containing product is beets.

7. The method of claim 1, wherein the solution is added directly to the sugar-containing product.

8. The method of claim 1, wherein the solution is added to a purified mixture resulting from the grinding of the sugar-containing product.

9. The method of claim 1, wherein the solution is added to press water collector.
